(19) United States
(12) Patent Application Publication
(10) Pub. No.: US 2013/0302439 A1
(43) Pub. Date: Nov. 14, 2013

(54) NEW USE OF FOOD COMPOSITIONS AND PRODUCTS COMPRISING OXIDES AND/OR HYDROXIDES

(71) Applicants: Alessandro Farinato, Mestre (IT); Dario Brunello, Mestre (IT); Luca Tombolan, Mestre (IT); Alfredo Guerrato, Mestre (IT)

(72) Inventors: Alessandro Farinato, Mestre (IT); Dario Brunello, Mestre (IT); Luca Tombolan, Mestre (IT); Alfredo Guerrato, Mestre (IT)

(21) Appl. No.: 13/990,593

(22) PCT Filed: Dec. 1, 2011
(86) PCT No.: PCT/IB2011/002903

§ 371 Date: Jul. 17, 2013

(30) Foreign Application Priority Data
Dec. 2, 2010 (IT) MI2010A002228

(51) Int. Cl. A61K 33/24; A61K 33/08; A61K 33/00

(52) U.S. Cl. CPC A61K 33/24; A61K 33/00; A61K 33/08
USPC 424/617; 424/724; 424/691; 424/690; 424/692

(57) ABSTRACT

The subject matter of the invention relates to the use of a composition comprising at least one oxide and/or hydroxide insoluble in an aqueous environment, preferably selected from silicon dioxide (SlO2), titanium dioxide (TlO2), aluminium oxide (AI2O3) and aluminium hydroxide (Al(OH)3), magnesium oxide (MgO), to reduce absorption of the ethanol ingested through the consumption of alcoholic beverages. The invention also comprises the use of food products comprising these mixtures for the aforesaid use.

The subject matter of the present invention is the use of a composition comprising at least one oxide and/or hydroxide insoluble in an aqueous environment, preferably selected from silicon dioxide (SiO₂), titanium dioxide (TiO₂), aluminium oxide (Al₂O₃) and aluminium hydroxide (Al(OH)₃), magnesium oxide (MgO), to reduce the absorption of ethanol ingested through the consumption of alcoholic beverages. The invention also comprises the use of food products comprising these mixtures for the aforesaid use.

### TECHNICAL BACKGROUND

Oxides and hydroxides such as silicon dioxide (SiO₂), aluminium oxide (Al₂O₃) and aluminium hydroxide (Al(OH)₃), titanium dioxide (TiO₂) and magnesium oxide (MgO) are compounds widely used in many technical fields and can also be ingested without causing undesirable and/or toxic effects. These compounds are easily available on the market.

It is known that a limited consumption of wine, especially red wine, such as two glasses per day, has beneficial effects on health, reducing LDL cholesterol, known as “bad” cholesterol, and increasing HDL cholesterol, i.e. the cholesterol defined as “good”, and also inhibiting platelet aggregation. Moreover, wine is a source of antioxidants, such as polyphenols, contains anti-carcinogenic substances and helps to keep arteries clear. It has in fact been observed that a moderate consumption of wine can help to reduce cardiovascular risk due to the beneficial properties of the nutrients contained therein.

Many beneficial properties have also been recognised in beer. In fact, just as for red wine, this beverage contains antioxidant substances, useful in reducing the risk of cardiovascular diseases. Drinking moderate amounts of beer, which also contains vitamins from the B group, also helps to control homocystein, the increase of which can lead to a predisposition towards cardiovascular diseases.

Drinking beverages such as wine and beer which, as stated, have a beneficial nutritional action on the organism (besides representing a pleasure for the consumer's palate, the reason for which they have become part of the daily diet) has however the drawback of also resulting in the intake of alcohol, in particular of ethanol which, as known, has many harmful effects on the health. In fact, it has been shown that the ingestion and consequent absorption of ethanol causes, i.a., cutaneous vasodilation, especially in the area in which flushing takes place (shoulders, neck and face), depressive effects on the central nervous system, arrhythmia and depresses the contractility of the cardiac muscle, in the long term leading to cardiomyopathy; ethanol can also cause an increase in blood pressure, irritates the gastro-intestinal mucous membranes and can interfere with the effect of prescription drugs acting on the central nervous system.

Basically, the beneficial effects deriving from beer and wine consumption, discussed above, tend to be cancelled by the simultaneous absorption of the ethanol contained in these beverages.

US2005/031708 describes the use of zeolites as detoxifiers to treat a variety of conditions, including alcohol intoxication. Zeolites are substances with a microporous crystalline structure characterized by an enormous quantity of empty volumes inside the crystals and are generally used as catalysts or as molecular sieves. To be administered to humans, zeolites must obviously be highly purified. In any case, US2005/031708 does not describe the use of zeolites to reduce ethanol absorption but only their detoxifying effect after excessive ethanol consumption.

### DESCRIPTION OF THE INVENTION

It has now been found that it is possible to reduce absorption of the ethanol ingested through alcoholic beverages habitually consumed with the diet, such as wine and beer, by the simultaneous consumption of compositions comprising some oxides and/or hydroxides.

Therefore, according to one of its aspects, the subject matter of the invention is the use of a composition comprising at least one oxide and/or hydroxide insoluble in an aqueous environment (hereinafter only “oxide(s) and/or hydroxide(s)”) to reduce absorption of the ethanol ingested through the consumption of alcoholic beverages.

“Oxide and/or hydroxide insoluble in an aqueous environment” is intended to indicate herein inorganic oxides and hydroxides which can be ingested by humans, therefore non-toxic and physiologically compatible oxides and hydroxides.

According to a preferred aspect, the subject matter of the invention is the use of a composition comprising one or more oxides and/or hydroxides selected from silicon dioxide (SiO₂), titanium dioxide (TiO₂), aluminium oxide (Al₂O₃) and aluminium hydroxide (Al(OH)₃), magnesium oxide (MgO), to reduce absorption of the ethanol ingested through the consumption of alcoholic beverages

Silicon dioxide (SiO₂), titanium dioxide (TiO₂), aluminium oxide (Al₂O₃) and aluminium hydroxide (Al(OH)₃), and magnesium oxide (MgO) are known compounds and are easily available on the market. These compounds are also commercially available in purified form, sufficiently pure and therefore suitable for administration to humans.

The composition according to the invention can comprise one or more of the aforesaid compounds, for example can comprise only one compound selected from silicon dioxide (SiO₂), titanium dioxide (TiO₂), aluminium oxide (Al₂O₃) and aluminium hydroxide (Al(OH)₃), magnesium oxide (MgO), or can comprise two or more of said compounds.

According to a preferred aspect of the invention, the composition preferably comprises one or more compounds selected from silicon dioxide (SiO₂), titanium dioxide (TiO₂) and aluminium oxide (Al₂O₃).

According to another preferred aspect of the invention, the composition comprises a single oxide, for example silicon dioxide (SiO₂) or titanium dioxide (TiO₂) or aluminium oxide (Al₂O₃).

According to an advantageous embodiment of the invention, the oxide employed is titanium dioxide (TiO₂). In fact, this oxide has proved to be particularly effective for the use according to the invention.

When more than one compound is present in the composition, the ratio between the various compounds can vary greatly without altering the ability of the composition to absorb alcohol.

The composition is preferably in solid form, for example in the form of powder, granules, tablet, capsule, etc.

Alternatively, the composition can be in the form of suspension or gel.

The composition according to the invention can contain only the oxides or hydroxides of the invention, or can also include any chemically neutral excipients, additives and/or flavouring agents, provided that these are edible and physiologically compatible.

The composition can also comprise absorbent charcoal (also called activated carbon). The composition of the invention can be in dosage unit form or in multi-dose form. Preferably, the composition is in dosage unit form and comprises from 2 to 8 g of oxide and/or hydroxide (when the composition comprises more than one oxide/hydroxide, the quantity refers in any case to the total weight of the mixture), advantageously from 4 to 6 g, even more advantageously approximately 5 g.

Another subject matter of the invention is the use of a food product for human consumption comprising a composition of one or more oxides and/or hydroxides, selected from silicon dioxide (SiO₂), titanium dioxide (TiO₂), aluminium oxide (Al₂O₃) and aluminium hydroxide (Al(OH)₃), magnesium oxide (MgO), to reduce absorption of the ethanol ingested through the consumption of alcoholic beverages. According to the present invention, the expression “food product” includes a food in the broadest sense of the term, including a functional food or food supplement, or also products defined as “medical devices” by current legislation.

By way of example, food product is intended as any substance or product whether processed, partially processed or unprocessed, intended to be, or reasonably expected to be ingested by humans. This includes drink, chewing gum and any substance, including water, intentionally incorporated into the food during its manufacture, preparation or treatment.

A food is generally defined as “functional” if it is demonstrated to have a beneficial effect on one or more target functions in the body, above and beyond the adequate and expected nutritional effects.

A “food supplement” is a product aimed at promoting the intake of certain nutritional substances not present in the foods of subjects who do not follow a correct and balanced diet.

A “medical device” is currently defined by Italian law (Legislative Decree no. 46/1997) as “an instrument, system, substance or other product used alone or in combination including the software used for its proper application, intended by the manufacturer to be used for human beings for the purpose of: diagnosis, prevention, monitoring, treatment or alleviation of disease; diagnosis, monitoring, treatment, alleviation of or compensation for an injury or handicap; investigation, replacement or modification of the anatomy or of a physiological process; control of conception. Food products for the use according to the invention include beverages, sweet and savoury bakery products, such as bread, pizza, focaccia, crackers, biscuits, cakes, snack cakes, sweets and the like; snacks such as crisps, dried fruit and nuts, bars, tortillas and the like; fresh or dry pasta, whether they are made of durum wheat flour, soft wheat flour, spelt flour, rice flour etc., or ready meals, sauces etc.

Therefore, the composition or the food product for use according to the invention, when consumed together with a moderate quantity of an alcoholic beverage, in particular wine or beer, leads to a significant reduction in the absorption of ethanol, thus allowing the organism to enjoy only the beneficial effects of these beverages.

The composition or the food product for use according to the invention can therefore be used in the daily diet, to limit the harmful effects of the ethanol contained in alcoholic beverages habitually consumed during meals, such as wine and beer.

Nonetheless, it is evident that the composition or the food product for use according to the invention can be consumed simultaneously to the consumption of alcoholic beverages other than wine and beer, such as aperitifs, digestive liqueurs and other beverages containing ethanol, preferably with a moderate alcohol content.

The composition or the food product for use according to the invention also permits a modest consumption of beverages having a moderate alcohol content also by subjects who, for various reasons, must follow an alcohol-free diet. In fact, due to the simultaneous consumption of the food product of the invention, these subjects can benefit from the consumption of the healthy components of the beverages described above, such as wine and beer, without putting their health at risk.

The composition for the use according to the invention can be prepared without any particular difficulties, as the components are practically inert and in any case compatible with one another and can be mixed together and/or with other substances (excipients, flavouring agents, etc.) without causing any problems.

The food product can be prepared according to normal techniques for preparing the selected food, mixing a suitable quantity of composition of the invention, as defined above, with the mixture of ingredients required to produce said food.

Thus, by way of example, to prepare a bakery product, the composition can be added to the mixture to be baked. Alternatively, the composition can be added to the flours required to prepare said mixture and the food product can be produced according to known techniques.

Flours for human use comprising the composition as defined above form a further subject matter of the invention. These flours include, for example, durum wheat flour, soft wheat flour, maize flour, rice flour etc.

To prepare a food product for the use according to the invention, the composition as defined above can be mixed with other components such as water, juices or fruit or vegetable extracts, flavours, sugar or sweeteners, salt, essential oils, etc.

By way of example, the food product in solid or liquid form can be in dosage unit or multi-dose form. When in dosage unit form, this food product can for example contain between 2 and 8 g, preferably between 4 and 6 g, advantageously approximately 5 g of the oxides/hydroxides as defined above or mixtures thereof.

The effect of the composition on limiting absorption of the ethanol ingested by a subject through the consumption of alcoholic beverages has been demonstrated by in vitro and in vivo assays. Details of these assays are set forth in the experimental section of the present description.

It has thus been observed that the quantities of composition required to counter the negative effects caused by the alcohol contained in alcoholic beverages, such as wine and beer, are in the order of 1 g of oxide/hydroxide (or mixtures thereof) for 5-6 ml of ethanol.

Therefore, by way of example, approximately 2 g of composition are able to inhibit absorption of the ethanol contained in approximately 100 ml, the equivalent of one glass of wine.

Likewise, approximately 2 g of composition are able to inhibit absorption of the ethanol contained in 330 ml of beer, the equivalent of one medium-sized bottle or one can.

By performing some simple calculations, within the grasp of those with average skill in the art, it is easy to determine the quantity of composition to use or to add to the food product according to the invention, in order to cancel the negative effects of a given quantity of alcoholic beverage habitually ingested.

These doses can naturally be varied on the basis of the needs of the subject consuming the composition for the use according to the invention.

Therefore, purely by way of illustration, it is possible, for example, to prepare bread or crackers to consume with meals, or liquid mixtures, comprising the composition as defined above in an amount from 2 to 8 g, for example 5 g, per ration, in such a manner as to be able to prevent the absorption of two glasses of wine, or of two cans of beer, consumed with the meal.

According to another aspect thereof, the subject matter of the invention is a method for limiting absorption of the ethanol ingested through the consumption of alcoholic beverages in a human subject, which comprises ingesting, prior to or simultaneously to the consumption of said alcoholic beverage, an effective quantity of a composition or a food product according to the invention.

### BRIEF DESCRIPTION OF THE DRAWINGS

FIGS. 1 and 2 show the decrease in alcoholic content obtained with different quantities of oxides/hydroxides, alone or mixed together.

### EXPERIMENTAL SECTION

### Example 1a

In Vitro Assay on the Ethanol-absorbing Properties of the Composition of the Invention

Tests were carried out on the absorption of ethanol from hydroalcoholic solutions by variable quantities of silicon dioxide (SiO₂), titanium dioxide (TiO₂), aluminium oxide (Al₂O₃) and aluminium hydroxide (Al(OH)₃), magnesium oxide (MgO) or mixtures thereof, at a temperature varying between 18 and 24° C.

The absorption readings were carried out on the basis of the decrease in alcohol concentration of the hydroalcoholic solutions after introduction of the composition.

The experiments were conducted by making up each time 500 ml of hydroalcoholic solution (distilled water taken to an alcohol content of 13% by the addition of 95% alcohol) and measuring the effective concentration with a series of Gay-Lussac aerometers with different scales. The solutions were contained in 500 ml glass cylinders in which the appropriate aerometer was made to float at the beginning and the concentration of alcohol in the water was then read. As it is known, the calibration of aerometers is based on the different densities of the solutions at a given temperature, the density of alcohol being 0.79 at 20° C.

Weighed quantities of composition were then introduced into the cylinder, and the variation in alcohol concentration was measured in the suspended sediment with the aerometer at different times, i.e. after 1′, 4′, 10′, 20′, 40′, 60′ and 90′. The variation in the density of the mixture (also in the absence of absorption) is negligible: in fact, introducing up to 15 g of composition in 500 ml of pure H₂O, there is no appreciable variation in volume.

All the readings regarding ethanol absorption by the composition were performed in the suspended sediment, i.e. in the suspension of the powder in the hydroalcoholic solution. The measurements of the alcoholic absorption at various time intervals were also performed after vigorous stirring of the mixture, in order to resuspend the sedimented inorganic matter.

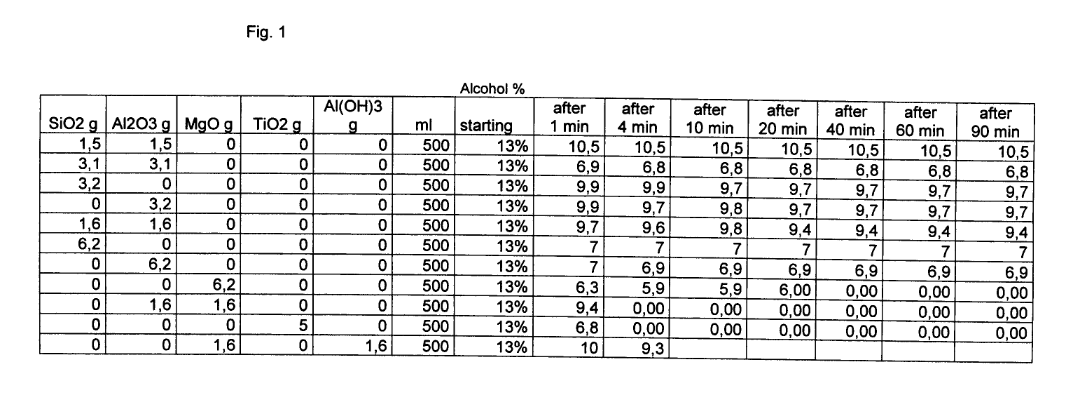

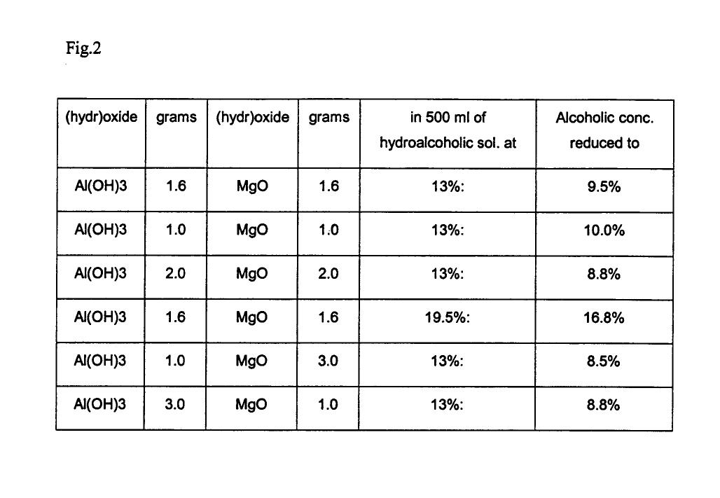

FIGS. 1 and 2 show the decrease in alcoholic content obtained with different quantities of oxides/hydroxides, alone or mixed together. As can be noted, the addition of the oxides/hydroxides caused a drastic reduction in the alcoholic content of the initial solution.

### Example 2

In Vivo Assay on the Ethanol-absorbing Properties of the Micronized Composition in Healthy Volunteers

In this phase of experimentation two healthy subjects were informed that we would measure the ability of the composition to absorb ethanol.

To perform the experiment, we conducted a medical examination on the two subjects to exclude metabolic disorders and simultaneous consumption of prescription drugs.

The two subjects, respectively 44 and 48 years of age, had the characteristics of healthy subjects, with an athletic build, with few fatty deposits located in region of the waist and hips, and no family history of alcohol or drug dependency.

The protocol used involved the consumption of 250 ml of wine with an alcohol content of 14%, consumed together with three slices of toast spread with peanut butter containing 5 grams of the composition (test) or not containing the composition (control), in a time of 20′,

Two test sessions were recorded: in the first the BAC (blood alcohol content) was measured with a breathalyser without prior consumption of the composition and in the second session after consumption of 5 grams of the composition.

The BAC measurements were taken at the following times:

1) Measurement 20′ after finishing the alcoholic beverage

2) Measurement 40′ after finishing the alcoholic beverage

3) Measurement 50′ after finishing the alcoholic beverage

4) Measurement 65′ after finishing the alcoholic beverage

5) Measurement 80′ after finishing the alcoholic beverage

The BAC was measured in the same subject after consumption of the alcoholic beverage (wine at 14°) without prior consumption of the composition and after having consuming 5 grams of composition prior to consumption of the alcoholic beverage, in the form indicated above.

Two separate test sessions were conducted, at a distance of three days from each other.

The test provided relevant data correlated to the ability of the composition contained in a food to absorb ethanol.

### Examples 3-12

Dosage units according to the invention comprise, as alcohol absorbing agent:

(TiO₂), (Al₂O₃) and aluminium hydroxide (Al(OH)₃), magnesium oxide (MgO),

| Example | Composition |
|---|---|
| 3 | 5 g of silicon dioxide (SiO₂) |
| 4 | 5 g of titanium dioxide (TiO₂) |
| 5 | 2.5 g of silicon dioxide (SiO₂₎ |
| | and 2.5 g of titanium dioxide |
| | (TiO₂) |
| 6 | 2.5 g of aluminium oxide (Al₂O₃) |
| | and 2.5 g of titanium dioxide |
| | (TiO₂) |
| 7 | 1.6 g of silicon dioxide (SiO₂), |
| | 1.6 g of aluminium oxide (Al₂O₃) |
| | and 1.6 g of titanium dioxide (TiO₂) |
| 8 | 2.5 g of aluminium oxide (Al₂O₃) |
| | and 2.5 g of silicon dioxide (SiO₂) |
| 9 | 2.5 g of silicon dioxide (SiO₂) and |
| | 2.5 g of magnesium oxide (MgO) |
| 10 | 2.0 g of titanium dioxide (TiO₂), 1.5 |
| | g of aluminium oxide (Al₂O₃) and |
| | 2.0 g of magnesium oxide (MgO) |
| 11 | 1.5 g of aluminium oxide (Al₂O₃) |
| | and 1.5 g of titanium dioxide (TiO₂) |
| | and 1.5 g of aluminium hydroxide |
| | (Al(OH)₃) |
| 12 | 1 g of silicon dioxide (SiO₂), 1 g of |
| | aluminium oxide (Al₂O₃), 1 g of |
| | aluminium hydroxide (Al(OH)₃), |
| | 1 g of titanium dioxide (TiO₂) and 1 g |
| | of magnesium oxide (MgO) |

1. The use of a composition comprising at least one oxide and/or hydroxide insoluble in an aqueous environment, to reduce the absorption of ethanol ingested through the consumption of alcoholic beverages.

2. The use according to claim 1, characterized in that said at least one oxide and/or hydroxide is selected from silicon dioxide (SiO₂), titanium dioxide (TiO₂), aluminium oxide (Al₂O₃), aluminium hydroxide (Al(OH)₃), magnesium oxide (MgO) and mixtures thereof.

3. The use according to claim 2, characterized in that said at least one oxide and/or hydroxide is selected from silicon dioxide (SiO₂), titanium dioxide (TiO₂), aluminium oxide (Al₂O₃) and mixtures thereof.

4. The use according to claim 1, characterized in that said composition comprises only one oxide and/or hydroxide.

5. The use according to claim 1, characterized in that said composition comprises two or more oxides and/or hydroxides.

6. The use according to claim 1, characterized in that said composition is in the form of dosage unit and comprises from 2 to 8 g of oxides and/or hydroxides and/or mixtures thereof.

7. The use according to claim 6, characterized in that said composition is in the form of dosage unit and comprises from 4 to 6 g of oxides and/or hydroxides and/or mixtures thereof.

8. The use according to claim 1, characterized in that said composition is in the form of powder, granules, tablet, capsule, suspension or gel.

9. The use of a food product for human use comprising the composition according to claim 1 to reduce absorption of the ethanol ingested through the consumption of alcoholic beverages.

10. The use according to claim 9, characterized in that said food product is selected from beverages, sweet and savoury bakery products, bread, pizza, focaccia, crackers, biscuits, cakes, snack cakes, sweets, snacks, crisps, dried fruit and nuts, bars, tortillas, fresh or dry pasta, ready meals, sauces.
